(19) United States
(12) Patent Application Publication
(10) Pub. No.: US 2023/0277807 A1
(43) Pub. Date: Sep. 7, 2023

(54) MICROCATHETER WITH MULTISTRAND BRAID DESIGN

(71) Applicant: Boston Scientific Medical Device Limited, Galway (IE)

(72) Inventors: ANSHUL CHABRA, Delhi (IN); JAHNAVI KONDURU, New Delhi (IN); RAHUL JAIN, Meerut (IN); RUPESH KUMAR PAHWA, Gurugram (IN); SHUBHAM AGRAWAL, Durg (IN)

(73) Assignee: Boston Scientific Medical Device Limited, Galway (IE)

(21) Appl. No.: 18/117,897

(22) Filed: Mar. 6, 2023

Related U.S. Application Data
(60) Provisional application No. 63/317,470, filed on Mar. 7, 2022.

(51) Int. Cl. A61M 25/00

(52) U.S. Cl. CPC A61M 25/005; A61M 25/0045; A61M 2025/0042; A61M 2025/0059

(57) ABSTRACT

The microcatheter may include an elongated shaft having a distal end and a proximal end. The elongated shaft may include an outer layer which may be formed from a polymer, a middle layer which may be formed of a braid having a plurality of strands having four filaments each, wherein the four filaments may be a round metal with an outer diameter of less than 0.0009”. The braid may achieve at least a 70% surface area coverage. The elongated shaft may include an inner layer which may be formed from a polymer, and a distal polymeric tip which may be attached to the distal end of the elongated shaft.

### CROSS-REFERENCE TO RELATED APPLICATIONS

This application claims the benefit of US Prov. Pat. App. No. 63/317,470, filed Mar. 7, 2022, titled MICROCATHETER WITH MULTISTRAND BRAID DESIGN, which is incorporated herein by reference.

### TECHNICAL FIELD

The present invention relates generally to catheters for delivery of therapeutic agents or devices to a site within a body lumen. More particularly, the present invention is directed to microcatheters used to navigate the vascular system.

### BACKGROUND

A variety of intravascular catheters are known, including small diameter catheters having a central lumen therethrough that are configured for use in smaller vasculature. Such catheters are known as microcatheters. Microcatheters are typically highly flexible and thin-walled, which can result in limited torque transfer from proximal hub to distal tip, reduce kink resistance, and difficulty pushing through tortuous vasculature. Microcatheters may also have limited ability to withstand bursting when fluid is passed through the central lumen, particularly at locations where the microcatheter is subject to kinking or tight angle turns. A need remains for improved microcatheters that increase torque transfer, burst pressure resistance, pushability, and kink resistance.

There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a microcatheter. The microcatheter may include an elongated shaft having a distal end and a proximal end. The elongated shaft may include an outer layer which may be formed from a polymer, a middle layer which may be formed of a braid having a plurality of strands including four filaments each, wherein the four filaments may be a round or other shape elongate metal piece, with an outer diameter of less than 0.0009”(0.023 mm). The braid may achieve at least a 70% surface area coverage on the inner polymer layer. The elongated shaft may include an inner layer which may be formed from a polymer, and a distal polymeric tip which may be attached to the distal end of the elongated shaft. Some examples may have strands which consist of exactly four filaments, each of which is a round metal.

Alternatively or additionally to any of the embodiments above, the braid may be formed in a two over, two under configuration.

Alternatively or additionally to any of the embodiments above, at least one strand may comprise at least one stainless steel filament and at least one tungsten filament.

Alternatively or additionally to any of the embodiments above, the filaments may have an outer diameter of 0.00075” (0.019 mm).

Alternatively or additionally to any of the embodiments above, the elongated shaft may have an outer diameter of less than 3 French, and an inner diameter in the range of about 0.025“ to about 0.027“ (about 0.64 mm to about 0.69 mm).

Alternatively or additionally to any of the embodiments above, the elongated shaft may provide a torque response of at least 0.9:1.

Alternatively or additionally to any of the embodiments above, the elongated shaft may provide a torque response of at least 0.95:1.

Alternatively or additionally to any of the embodiments above, the braid may be in the range of about 100 to about 140 picks per inch (PPI).

Alternatively or additionally to any of the embodiments above, the braid may be in the range of about 120 PPI.

Alternatively or additionally to any of the embodiments above, the braid may comprise 12 to 20 strands each having four metal filaments having an outer diameter of about 0.0006 to 0.0009”(about 0.015 mm to about 0.023 mm).

Alternatively or additionally to any of the embodiments above, the braid may have 16 strands.

Alternatively or additionally to any of the embodiments above, the filaments may have an outer diameter about of 0.00075”(about 0.019 mm).

Alternatively or additionally to any of the embodiments above, the tip may be formed of a polymer with a shore hardness less than 40D.

Alternatively or additionally to any of the embodiments above, the elongated shaft may have a burst pressure in the range of 1000 to 3000 psi.

Alternatively or additionally to any of the embodiments above, the elongated shaft may have a burst pressure greater than 1200 psi.

In another example, a microcatheter may include an elongated shaft having a distal end and a proximal end. the elongated shaft may include an outer layer which may be formed of a polymer, a middle layer which may be formed of a braid having a plurality of strands having four filaments each being a round metal with an outer diameter of 0.00075” (about 0.019 mm), the braid achieving at least a 70% surface area coverage, and an inner layer which may be formed of a polymer. The braid may be formed in a two over, two under configuration. A distal polymeric tip may be attached to the distal end of the elongated shaft, and the elongated shaft may provide a torque response of at least 0.9:1.

Alternatively or additionally to any of the embodiments above, at least one strand may comprise at least one stainless steel filament and at least one tungsten filament.

Alternatively or additionally to any of the embodiments above, the braid may be in the range of about 100 to about 140 picks per inch (PPI).

Alternatively or additionally to any of the embodiments above, the braid may be in the range of about 120 PPI.

Alternatively or additionally to any of the embodiments above, the braid may comprise 12 to 20 strands each having four metal filaments having an outer diameter of about 0.0006 to 0.0009” (about 0.015 mm to about 0.023 mm).

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of a microcatheter in accordance with an embodiment of the invention;

FIG. 2 is a partial view of an elongated shaft of a microcatheter in accordance with an embodiment of the invention, illustrating an outer layer, a middle layer, and an inner layer;

FIG. 3 is a partial longitudinal section view of a portion of the elongated shaft as shown in FIG. 2;

FIG. 4 is a partial view of a braid of an elongated shaft of a microcatheter in accordance with an embodiment of the invention;

FIG. 5 is an illustrative graph comparing burst pressure of a microcatheter manufactured with a three-wire strand versus a four-wire strand;

FIG. 6 is an illustrative graph comparing kink radii of a microcatheter made with a three-wire strand versus a four-wire strand;

FIG. 7 is an illustrative graph comparing a three-point bend load of a microcatheter made with a three-wire strand versus a four-wire strand;

FIG. 8 is an illustrative graph comparing an axial load of a microcatheter made with a three-wire strand versus a four-wire strand; and

FIG. 9 is an illustrative graph depicting a torque response of a microcatheter in accordance with an embodiment of the invention.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. The recitation of numerical ranges by endpoints includes all numbers within that range (e.g., 1 to 5 includes, 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

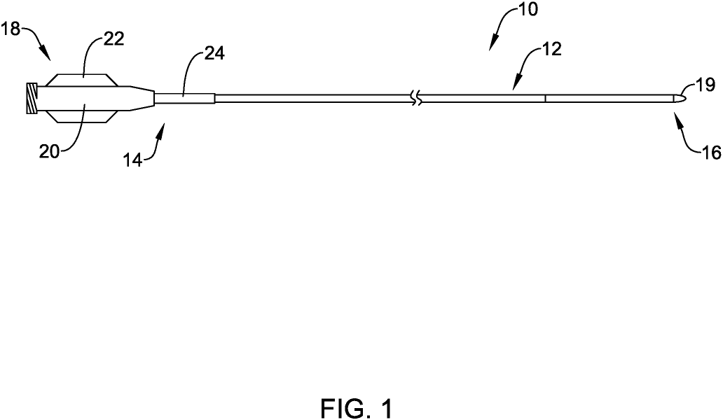

Microcatheters are used for a variety of therapeutic treatments such as diagnosis of vascular complications, delivery of an embolic treatment, and intravascular mapping. FIG. 1 is an example of such a microcatheter 10. As shown in FIG. 1, the microcatheter 10 may include an elongated shaft 12 having a distal end 16 and a proximal end 14. The microcatheter 10 may have a length that is in the range of about 50 to 200 centimeters and may have an outer diameter (OD) that is less than 3 French. In some cases, the microcatheter 10 may have an OD of 1.7 French, 2 French, 2.5 French, 2.8 French, or any other suitable outer diameter. In some cases, the microcatheter 10 may include an OD of about 2.6 French at the distal end 16, and an OD of about 2.8 French at the proximal end 14. In some cases, the microcatheter 10 may have an inner diameter (ID) of about 0.025 inches to 0.027 inches. In other cases, the microcatheter 10 may have an ID of about 0.02 inches, 0.03 inches, or any other suitable inner diameter. These sizes may vary depending on particular usage.

In some cases, a distal tip 19 may be connected to or disposed about the distal end 16 of the elongated shaft 12. The distal tip 19 may be a polymeric distal tip 19, which may be formed from an elastomer (e.g., Pebax®), a thermoplastic polymer, or any other suitable polymer. The distal tip may be formed of a softer material than other portions of the outer shaft of the microcatheter, such as by using a polymer or elastomer having a shore hardness of less than 40D. In some cases, the distal tip 19 may be formed from a polymer with a shore hardness of about 35D. In some cases, the distal tip 19 may have a length of about 1 millimeter (mm). In other cases, the distal tip 19 may have a length of about 1.5 mm, 1.3 mm, 1.7 mm, or any other suitable length. The distal tip 19 may include an outer diameter that is sized to match the OD of the microcatheter 10. In some cases, the distal tip 19 may include an OD of 1.7 French, 2 French, 2.5 French, 2.8 French, or any other suitable diameter. Some examples may include a transition region having varying hardness, such as using Pebax in varying hardnesses (for example, 75D to 63D to 55D to 45D from proximal to distal, to a 35D tip). These are just examples.

In some cases, as shown, a hub and strain relief assembly 18 may be connected to or disposed about the proximal end 14 of the elongated shaft 12. The hub and strain relief assembly 18 may include a main body portion 20, a pair of flanges 22 designed to improve gripping, and a strain relief 24 intended to reduce kinking. The hub and strain relief assembly 18 may be a conventional design and attached by conventional techniques.

As will be discussed, the elongated shaft 12 may be adapted to provide enhanced torque response, meaning that a particular rotation made at the proximal end 14 will be communicated to the distal end 16. In some cases, the elongated shaft 12 may be adapted to provide sufficient torque response such that a particular rotation made at the proximal end 14 will provide a torque response of at least 0.9:1 (e.g., ninety (90) percent). As an example, a 90-degree rotation made at the proximal end 14 will correlate to a rotation made at the distal end 16 that is around 81 degrees. In some cases, the elongated shaft 12 may provide a torque response of at least 0.95:1 (e.g., ninety-five (95) percent). As an example, a 90-degree rotation made at the proximal end 14 will correlate to a rotation made at the distal end 16 that is around 85.5 degrees. In other cases, the elongated shaft 12 may provide a torque response of at least 0.98:1 (e.g., ninety-eight (98) percent) at the distal end 16. As an example, a 360-degree rotation made at the proximal end 14 will correlate to a rotation made at the distal end 16 that is around 356 degrees. These are just examples.

In some cases, the microcatheter 10 may be considered as being an over the wire (OTW) microcatheter, configured for passing the entire length thereof over a guidewire. It will be appreciated that in cases in which the microcatheter 10 is instead a rapid exchange microcatheter, a side port may be provided to allow the guidewire to exit the microcatheter lumen distal of the proximal hub and strain relief assembly 18, such as shown in U.S. Pat. 8,636,714, the disclosure of which is incorporated herein by reference.

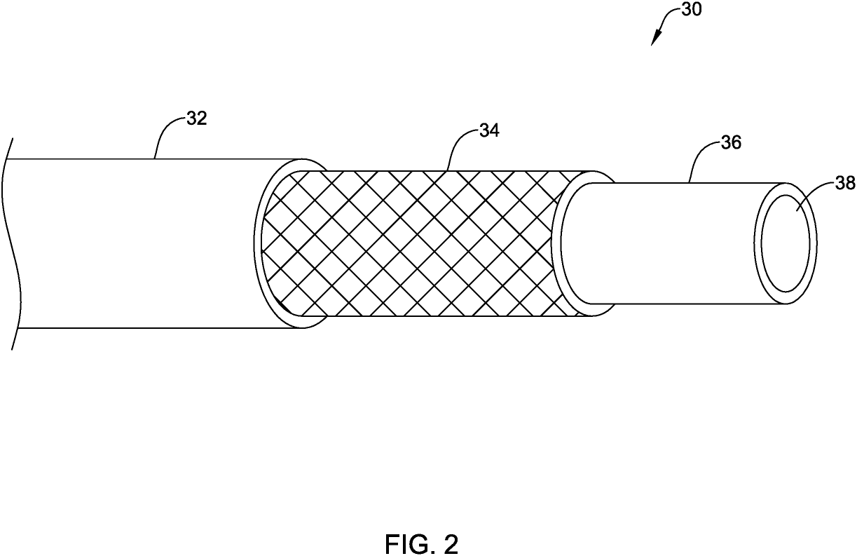

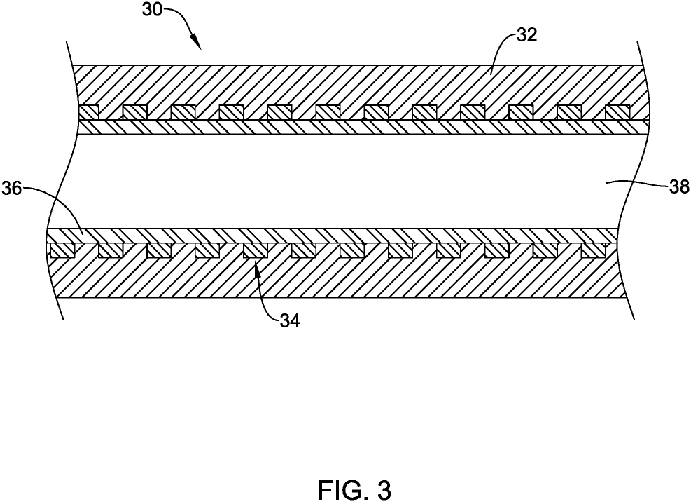

As noted, the elongated shaft 12 may be adapted to provide sufficient torque response. FIG. 2 is a partial view of an elongated shaft 30 of a microcatheter in accordance with an embodiment of the invention. FIG. 3 is a partial longitudinal section view of a portion of the elongated shaft 30, as shown in FIG. 2. The elongated shaft 30 may be an example of the elongated shaft 12 shown in FIG. 1. It will be appreciated that the illustrated portion of the elongated shaft 30 could correspond to essentially any portion of the elongated shaft 12 shown in FIG. 1. The elongated shaft 30 may include an outer layer 32, a middle layer 34, an inner layer 36, and a lumen 38 extending therethrough. The lumen 38 may be considered as a guidewire lumen, as well as an injection lumen, or the like. As an example, in use, a practitioner may insert the microcatheter (e.g., microcatheter 10) over a guidewire (not shown). Once the target vessel is reached, the guidewire may be removed and a fluid may be injected at the target site through the lumen 38.

The inner layer 36 of the elongated shaft 30 may be formed from or include a coating of a material having a suitably low coefficient of friction. Examples of suitable materials may include polytetrafluoroethylene (PTFE). The inner layer 36 may be dimensioned to define the lumen 38, having an appropriate inner diameter to accommodate its intended use, such as high flow rates. In some cases, the inner layer 36 may define a lumen 38 having a diameter of about 0.025 inches to 0.027 inches, or larger or smaller.

The outer layer 32 may be formed from a polymer that may provide the desired flexibility and strength. In some cases, the outer layer 32 may be formed from a nylon polymer, a thermoplastic polymer, elastomeric polyamides, or any other suitable polymer. The outer layer 32 may be dimensioned to define the outer diameter of the elongated shaft 30. In some cases, as discussed with reference to FIG. 1, the outer diameter of the elongated shaft 30 may be less than 3 French. In some cases, the outer layer 32 may have an OD of 1.7 French, 2 French, 2.5 French, 2.8 French, or any other suitable outer diameter. The outer layer 32 may comprise a plurality of segments to provide varying characteristics (lubricity, flexibility, outer diameter, etc.) along the length of the assembly. Further, more than one outer layer 32 may be used at locations as desired.

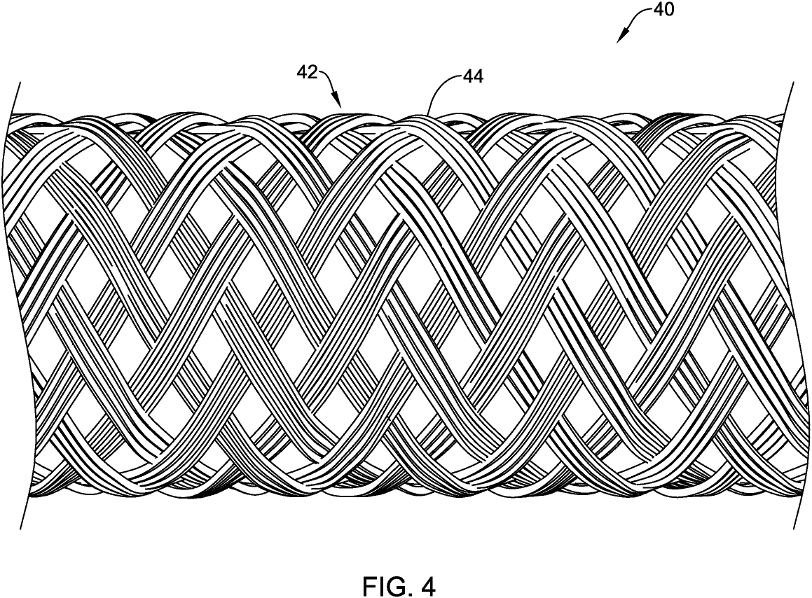

The middle layer 34 may be positioned between the outer layer 32 and the inner layer 36, and may be formed of a braid. The middle layer 34 may be considered to be a reinforcing layer that increases the torque response of the elongated shaft 30. The middle layer 34 may be formed of any suitable material, such as stainless steel, tungsten, gold, titanium, silver, copper, platinum, or nitinol. In some cases, the middle layer 34 may be formed from a non-metallic material such as polymer fibers, glass fibers, or liquid crystal polymer (LCP) fibers. The middle layer 34 may be formed using a variety of different weave patterns, such as a three-over-three, a four-over-four, or the like. In some cases, the middle layer 34 may be formed using a two over, two under configuration, as will be discussed further with reference to FIG. 4.

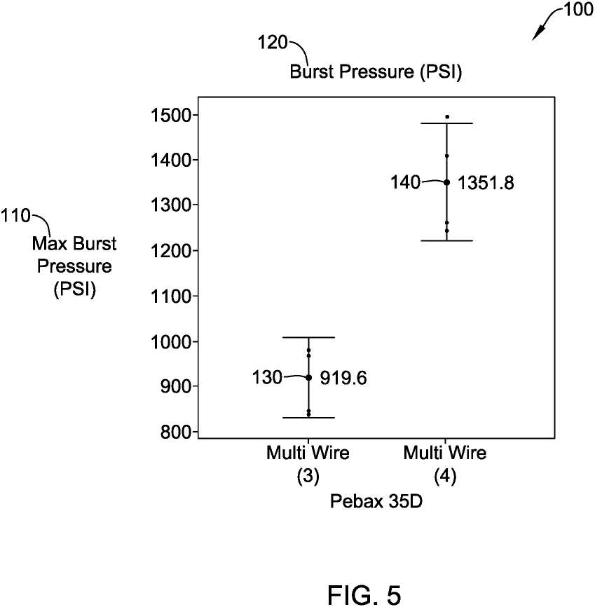

FIG. 4 is a partial view of a braid 40 of an elongated shaft (e.g., elongated shaft 30). As previously discussed, with reference to FIGS. 2 and 3, the braid 40 may form the middle layer (e.g., middle layer 34) of the elongated shaft (e.g., elongated shaft 30). The braid 40 may be formed from a plurality of strands 42, wherein each strand may be formed from a plurality of filaments 44. The braid 40 may comprise twelve (12) to twenty (20) strands 42. In some cases, the braid 40 may comprise sixteen (16) strands 42. The braid 40 may be in the range of about 100 to about 140 picks per inch (PPI). In some cases, the braid 40 may be in the range of about 120 PPI. In some examples, the braid 40 may achieve at least a seventy (70) percent coverage of the surface area of the inner layer 36, which thereby provides the elongated shaft (e.g., elongated shaft 30) with a high burst pressure performance, which will be further discussed with reference to FIG. 5. In some cases, the braid 40 may achieve at least a sixty (60) percent coverage, an eighty (80) percent coverage, a ninety (90) percent coverage, or any other suitable percentage coverage of the surface area of the inner layer 36.

In some cases, as shown in FIG. 4, the plurality of strands 42 may include four filaments 44 each. The filaments 44 may be formed of any suitable material, such as stainless steel, tungsten, gold, titanium, silver, copper, platinum, or nitinol. In some cases, at least one strand 42 may include at least one stainless steel filament 44 and at least one tungsten filament 44. In other cases, at least one strand 42 may include two stainless steel filaments 44 and two tungsten filaments 44. In other cases, at least one strand 42 may be formed from four stainless steel filaments 44 and a second strand 42 may be formed from four tungsten filaments 44. Each strand 42 may be formed from any suitable combination of filaments 44, as desired. Some example strands may have five or more filaments. Other examples may have strands consisting of exactly four filaments.

In some cases, each strand 42 is formed from four filaments 44 stacked together and braided in a two over, two under configuration. Testing illustrated below shows that the four filament 44 braid in a two over, two under configuration may provide the elongated shaft (e.g., elongated shaft 30) with a greater torque response, and allow for greater pushability, thereby reducing the number of kinks that may form in the elongated shaft.

The filaments may have a round cross-sectional shape in some examples. Alternatively, the filaments may include a rectangular, oval, or any other suitable cross-sectional shape. Each of the filaments 44 may include an outer diameter of less than 0.0009 inches. In some cases, each filament 44 may include an outer diameter of 0.00085 inches, 0.0008 inches, 0.00075 inches, 0.0006 inches, or any other suitable diameter. Filaments may, for example, have an outer diameter in the range of about 0.0006 inches to about 0.0009 inches, or in the range of about 0.00075 inches to about 0.00085 inches. Each filament may be the same size and shape, or the filaments may be of different sizes and shapes. For example, a strand 42 may include two larger tungsten filaments 44 and two smaller stainless-steel filaments 44.

FIG. 5 is an illustrative graph 100 depicting a difference in a burst pressure 120 between a microcatheter shaft having a reinforcing layer with three-wire strand and a four-wire strand, each in a two over, two under configuration. As shown in FIG. 5, the burst pressure 120, which is measured in pounds per square inch (PSI), for a microcatheter including a braid formed from three filaments was compared to a microcatheter including a braid formed from four filaments each in a two over, two under configuration. A maximum burst pressure 110 may be the maximum pressure that can be applied to the microcatheter before the failure (e.g., burst or leak) of a microcatheter wall. As shown, the average maximum burst pressure 110 for the microcatheters including the braid formed from three filaments was 919.6 PSI, as referenced at 130. The average maximum burst pressure 110 for the microcatheters including the braid formed from four filaments was 1351.8 PSI, as referenced at 140. As discussed, the braid formed from four filaments may achieve at least a seventy (70) percent coverage of the surface area of the inner layer, thereby increasing the burst pressure of the microcatheter. In some cases, the braid may achieve at least a sixty (60) percent coverage, an eighty (80) percent coverage, a ninety (90) percent coverage, or any other suitable percentage coverage of the surface area of the inner layer. Thus, it may be more desirable to utilize a microcatheter including a braid formed from four filaments (e.g., braid 40) as the higher burst pressure may allow for a higher flow rate.

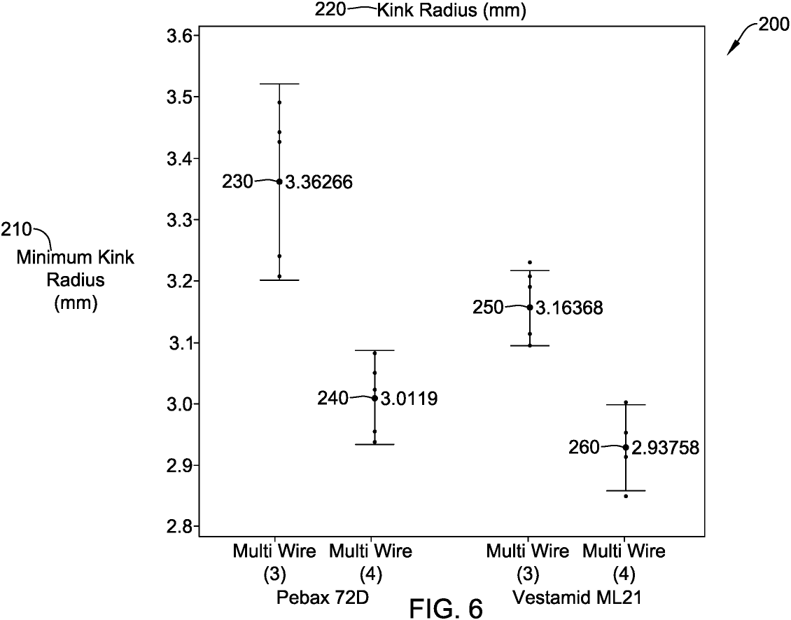

FIG. 6 is an illustrative graph 200 comparing kink radii of a microcatheter (e.g., microcatheter 10) made with a three-wire strand versus a four-wire strand. The kink radius is the minimum radius at which a lumen of a microcatheter may collapse, thereby forming a kink. As shown in FIG. 6, the kink radius 220, which is measured in millimeters (mm), for a microcatheter including a braid formed from three filaments was compared to a microcatheter including a braid formed from four filaments, each in a two over, two under configuration. Further, FIG. 6 illustrates a comparison of the kink radius 220 of a microcatheter formed from a three-wire braid and a four-wire braid formed from Pebax® 72D versus a microcatheter formed from a three-wire braid and a four-wire braid formed from Vestamid® ML21. As shown, the minimum kink radius 210 for the microcatheter, formed from Pebax® 72D, including the braid formed from three filaments includes a minimum kink radius 210 of 3.36266 mm, as referenced by 230. The minimum kink radius 210 for the microcatheter, formed from Pebax® 72D, including the braid formed from four filaments includes a minimum kink radius 210 of 3.0119 mm, as referenced by 240. The minimum kink radius 210 for the microcatheter, formed from Vestamid® ML21, including the braid formed from three filaments includes a minimum kink radius 210 of 3.16368 mm, as referenced by 250. The minimum kink radius 210 for the microcatheter, formed from Vestamid® ML21, including the braid formed from four filaments includes a minimum kink radius 210 of 2.93758 mm, as referenced by 260. As discussed, the braid formed from four filaments may achieve at least a seventy (70) percent coverage of the surface area of the inner layer, thereby minimizing the kink radius of the microcatheter. Thus, it may be more desirable to utilize a microcatheter including a braid formed from four filaments (e.g., braid 40).

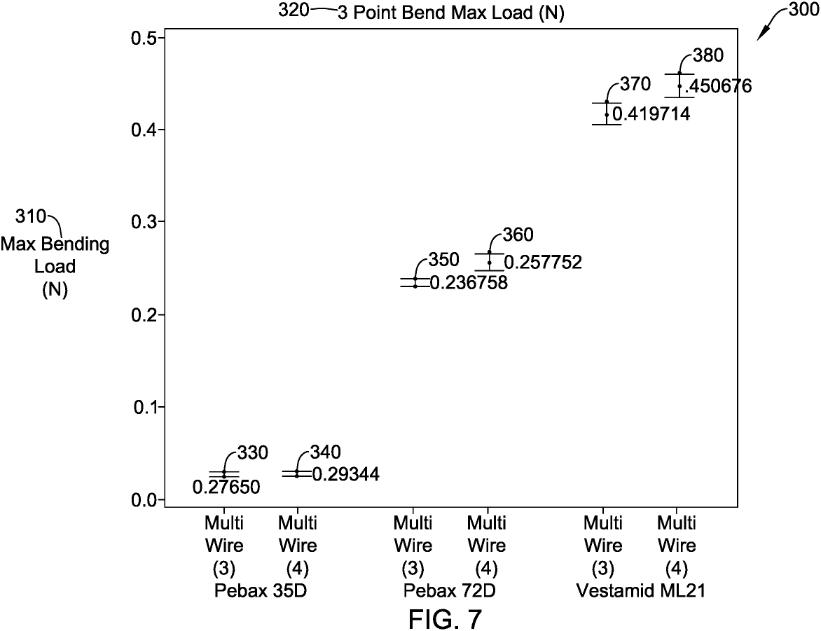

FIG. 7 is an illustrative graph 300 comparing a three-point bend load of a microcatheter (e.g., microcatheter 10) made with a three-wire strand versus a four-wire strand. A three-point bend maximum load 320 measures the bending stiffness, such as a flexibility, of an elongated shaft (e.g., elongated shaft 12) of the microcatheter (e.g., microcatheter 10). In some examples, a proximal portion of the microcatheter (e.g., proximal end 14) may include a bending stiffness that allows for a required flexibility when navigating tortuous vessels. Further, in some examples, a distal portion (e.g., distal end 16) of the microcatheter should be relatively more flexible so as to allow for better trackability, such as over a guidewire, for example. As with other graphs, the tested microcatheter shafts or assemblies were formed with braids having multifilament strands (having three or four filaments) in a two over, two under braid between inner and outer layers.

As shown in FIG. 7, the bend load (e.g., three-point bend maximum load 320, maximum bending load 310), which is measured in Newtons (N), for a microcatheter including a braid formed from three filaments was compared to a microcatheter including a braid formed from four filaments. Further, FIG. 7 illustrates a comparison of the bend load 320 of a microcatheter formed from a three-wire braid and a four-wire braid formed from Pebax® 35D versus a microcatheter formed from a three-wire braid and a four-wire braid formed from Pebax® 72D, and a microcatheter formed from a three-wire braid and a four-wire braid formed from Vestamid® ML21. As shown, a maximum bending load 310 for a microcatheter, formed from Pebax® 35D, including the braid formed from three filaments includes the maximum bending load 310 of 0.027650 N, as referenced by 330. The maximum bending load 310 for a microcatheter, formed from Pebax® 35D, including the braid formed from four filaments includes the maximum bending load 310 of 0.029344 N, as referenced by 340. The maximum bending load 310 of a microcatheter, formed from Pebax® 72D, including the braid formed from three filaments includes the maximum bending load 310 of 0.236758 N, as referenced by 350. The maximum bending load 310 for the microcatheter, formed from Pebax® 72D, including the braid formed from four filaments includes the maximum bending load 310 of 0.257752 N, as referenced by 360. The maximum bending load 310 for the microcatheter, formed from Vestamid® ML21, including the braid formed from three filaments includes the maximum bending load 310 of 0.419714 N, as referenced by 370. The maximum bending load 310 for the microcatheter, formed from Vestamid® ML21, including the braid formed from four filaments includes the maximum bending load 310 of 0.450676 N, as referenced by 380. Thus, it may be more desirable to utilize a microcatheter including a braid formed from four filaments (e.g., braid 40).

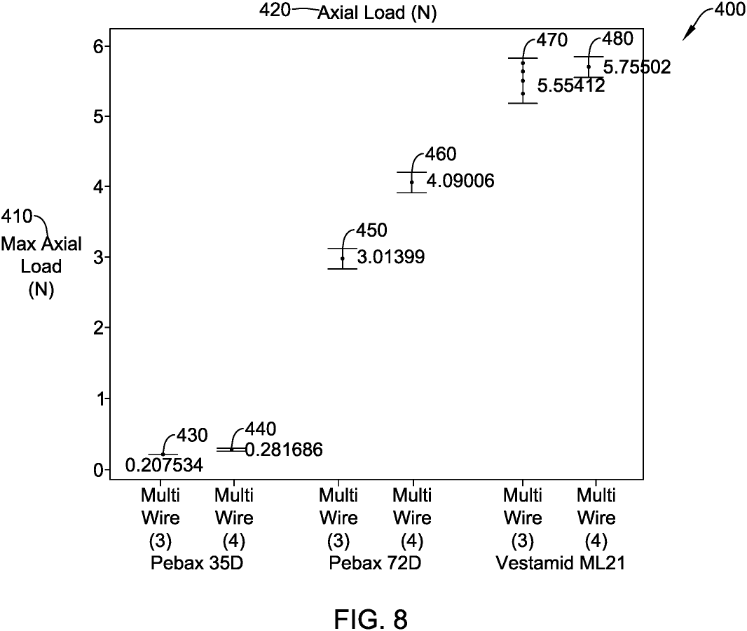

FIG. 8 is an illustrative graph 400 comparing an axial load of a microcatheter (e.g., microcatheter 10) made with a three-wire strand versus a four-wire strand. An axial load 420 measures the buckling strength, such as pushability, of an elongated shaft (e.g., elongated shaft 12) of the microcatheter (e.g., microcatheter 10). In some examples, a proximal portion of the microcatheter (e.g., proximal end 14) may include a higher axial stiffness for better pushability. In some examples, a distal portion (e.g., distal end 16) of the microcatheter should be flexible so as to allow for better trackability, such as over a guidewire, for example.

As shown in FIG. 8, the axial load 420, which is measured in Newtons (N), for a microcatheter including a braid formed from three filaments was compared to a microcatheter including a braid formed from four filaments. Further, FIG. 8 illustrates a comparison of the axial load 420 of a microcatheter formed from a three-wire braid and a four-wire braid formed from Pebax® 35D versus a microcatheter formed from a three-wire braid and a four-wire braid formed from Pebax® 72D, and a microcatheter formed from a three-wire braid and a four-wire braid formed from Vestamid® ML21. As shown, a maximum axial load 410 for a microcatheter, formed from Pebax® 35D, including the braid formed from three filaments includes the maximum axial load 410 of 0.207534 N, as referenced by 430. The maximum axial load 410 for a microcatheter, formed from Pebax® 35D, including the braid formed from four filaments includes the maximum axial load 410 of 0.281686 N, as referenced by 440. The maximum axial load 410 of a microcatheter, formed from Pebax® 72D, including the braid formed from three filaments includes the maximum axial load 410 of 3.01399 N, as referenced by 450. The maximum axial load 410 for the microcatheter, formed from Pebax® 72D, including the braid formed from four filaments includes the maximum axial load 410 of 4.09006 N, as referenced by 460. The maximum axial load 410 for the microcatheter, formed from Vestamid® ML21, including the braid formed from three filaments includes the maximum axial load 410 of 5.55412 N, as referenced by 470. The maximum axial load 410 for the microcatheter, formed from Vestamid® ML21, including the braid formed from four filaments includes the maximum axial load 410 of 5.75502 N, as referenced by 480. Thus, it may be more desirable to utilize a microcatheter including a braid formed from four filaments (e.g., braid 40).

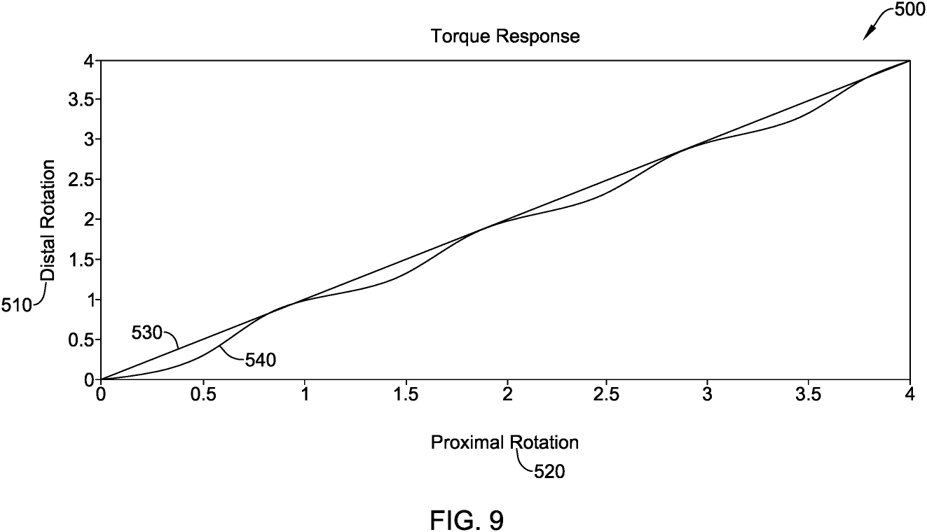

FIG. 9 is an illustrative graph 500 depicting a torque response of a microcatheter (e.g., microcatheter 10) in accordance with an embodiment of the invention. The tested microcatheter includes a two over, two under braid with 75% coverage comprised of strands each having four filaments, with each filament being a round metal wire with a 0.00075-inch outer diameter, having a 2.6F distal outer diameter and 2.8F proximal outer diameter, using an inner PTFE layer and a Vestamid® ML21, transitioning to a Pebax 35D distal tip, and having a total length of 135 cm. As shown in FIG. 9, line 530 represents a torque response of 1:1, meaning a proximal rotation 520 will correlate to a distal rotation 510 one hundred (100) percent. Line 540 represents a torque response of a microcatheter in accordance with an embodiment of the invention. As shown, the elongated shaft may be adapted to provide sufficient torque response such that a particular rotation made at the proximal end (e.g., proximal rotation 520) will provide a torque response of at least 0.9:1 (e.g., ninety (90) percent). In some cases, the proximal rotation 520 may provide a torque response of 0.98:1 (e.g., ninety-eight (98) percent).

The microcatheter 10, and various components thereof, may be manufactured according to essentially any suitable manufacturing technique including extruding, coextruding, molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A microcatheter comprising:
an elongated shaft having a distal end and a proximal end, the elongated shaft including:
an outer layer formed of a polymer;
an inner layer formed of a polymer; and
a middle layer formed of a braid having a plurality of strands including at least four metal filaments each with an outer diameter of less than 0.0009”, the braid achieving at least a 70% surface area coverage relative to an outer surface of the inner layer; and
a distal polymeric tip attached to the distal end of the elongated shaft.

2. The microcatheter of claim 1 wherein the braid is formed in a two over, two under configuration.

3. The microcatheter of claim 1 wherein at least one strand comprises at least one stainless steel filament and at least one tungsten filament.

4. The microcatheter of claim 1 wherein the filaments have an outer diameter of 0.00075”.

5. The microcatheter of claim 1 wherein the elongated shaft has an outer diameter of less than 3 French, and an inner diameter in the range of about 0.025” to about 0.027”.

6. The microcatheter of claim 1 wherein the elongated shaft provides a torque response of at least 0.9:1.

7. The microcatheter of claim 1 wherein the elongated shaft provides a torque response of at least 0.95:1.

8. The microcatheter of claim 1 wherein the braid is in the range of about 100 to about 140 picks per inch (PPI).

9. The microcatheter of claim 8 wherein the braid has in the range of about 120 PPI.

10. The microcatheter of claim 1 wherein the braid comprises 12 to 20 strands each having four metal filaments having an outer diameter of about 0.0006 to 0.0009”.

11. The microcatheter of claim 10 wherein the braid has 16 strands.

12. The microcatheter of claim 10 wherein the filaments have an outer diameter about of 0.00075”.

13. The microcatheter of claim 1 wherein each strand consists of four round metal filaments.

14. The microcatheter of claim 1 wherein the elongated shaft has a burst pressure in the range of 1000 to 3000 psi.

15. The microcatheter of claim 14 wherein the elongated shaft has a burst pressure greater than 1200 psi.

16. A microcatheter comprising:
an elongated shaft having a distal end and a proximal end, the elongated shaft including:
an outer layer formed of a polymer;
an inner layer formed of a polymer; and
a middle layer formed of a braid having a plurality of strands having four filaments each being a round metal with an outer diameter of 0.00075”, the braid achieving at least a 70% surface area coverage relative to the inner layer;
wherein the braid is formed in a two over, two under configuration; and
a distal polymeric tip attached to the distal end of the elongated shaft;
wherein the elongated shaft provides a torque response of at least 0.9:1.

17. The microcatheter of claim 16 wherein at least one strand comprises at least one stainless steel filament or at least one tungsten filament.

18. The microcatheter of claim 16 wherein the braid is in the range of about 100 to about 140 picks per inch (PPI).

19. The microcatheter of claim 18 wherein the braid is in the range of about 120 PPI.

20. The microcatheter of claim 16 wherein the braid comprises 12 to 20 strands each having four metal filaments having an outer diameter of about 0.0006 to 0.0009“.
